(19) United States
(12) Patent Application Publication
(10) Pub. No.: US 2026/0216022 A1
(43) Pub. Date: Jul. 30, 2026

(54) COMPOSITIONS COMPRISING COMPOUNDS HAVING RETINOL-LIKE ACTIVITY AND A PROCESSED OAT INGREDIENT

(71) Applicant: JOHNSON & JOHNSON CONSUMER INC., Skillman, NJ (US)

(72) Inventors: Ramine Parsa, Lawrenceville, NJ (US); Wen-Hwa Ting Li, Summit, NJ (US); Daphne Meza, Lansdale, PA (US); Ruchi Patel, Summit, NJ (US); Tobias Futterer, Summit, NJ (US)

(21) Appl. No.: 19/147,519

(22) PCT Filed: Jan. 11, 2024
(86) PCT No.: PCT/IB2024/050303

§ 371 Date: Jul. 11, 2025

Related U.S. Application Data
(60) Provisional application No. 63/479,433, filed on Jan. 11, 2023.

(51) Int. Cl. A61K 8/36; A61K 8/9789; A61K 8/9794; A61Q 19/02; A61Q 19/08

(52) U.S. Cl. CPC A61K 8/36; A61K 8/9789; A61K 8/9794; A61Q 19/02; A61Q 19/08; A61K 2800/85

(57) ABSTRACT

Provided are skincare compositions comprising: a compound having retinol-like activity and a processed oat ingredient. Also provided are methods for treating skin comprising topically applying these compositions. Also provided are skincare compositions comprising 3-(4-farnesyloxyphenyl)-propionic acid; and a processed oat ingredient selected from the group consisting of fermented oat, colloidal oat, oat extract, oat oil, and combinations thereof, wherein the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

### FIELD

The present invention generally relates to compositions suitable for use on skin and particularly compositions comprising compounds having retinol-like activity and a processed oat ingredient.

### BACKGROUND

The human skin is subject to certain aging processes, some of which are attributable to intrinsic processes (e.g. chronoaging) and some of which are attributable to exogenous factors (e.g. photo-aging). In addition, temporary or even lasting changes to the skin can occur, such as acne, greasy or dry skin, keratoses, rosacea, light-sensitive, inflammatory, erythematous, and allergic or autoimmune-reactive reactions, such as dermatosis and photodermatosis.

The consequences of the above-mentioned ageing processes can include thinning of the skin, weaker interlacing of epidermis and dermis, and a reduction in the number of cells and the supplying blood vessels. These consequences are often undesirable, and individuals suffering from these issues will look to topical treatments to address them.

Retinoids have been used for treating skin conditions caused by intrinsic aging, exogenous factors, acne or skin diseases. However, despite the beneficial effects of retinoid treatment, its benefits are limited due to skin irritation of retinoids. These side effects can restrict the use of retinoids, and particularly so for individuals having sensitive skin.

To date, the search for alternative compounds to replace retinoids, and particularly for individuals having sensitive skin, has produced limited success in treating skin conditions associated with aging, such as skin atrophy, acne, photo-aging, and in reducing the appearance of wrinkles, fine lines, stretch marks, or cellulite.

Accordingly, there is a need for alternatives to traditional retinoids which are efficacious but also gentle enough on the skin to be well-tolerated.

### SUMMARY

Accordingly, one aspect of the invention pertains to a skincare composition comprising:
a compound of Formula I:

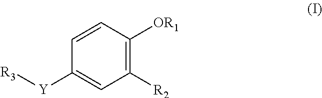

wherein:
   R₁ is selected from the group consisting of C₁-C₂₀ alkyl, C₂-C₂₀ alkenyl, C₂-C₂₀ alkynyl, and C₃-C₈ cycloalkyl or aryl;
   R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
   R₃ is selected from —CO₂H, —CO₂R₄ or an isosteric equivalent of a carboxy group, wherein R⁴ is C₁-C₆ alkyl, C₂-C₆ alkenyl, C₃-C₈ cycloalkyl or aryl; and Y is —(CH₂—CH₂)—, —(CH═CH)—, or —(C≡C)—;
   or a cosmetically acceptable salt thereof; and
   a processed oat ingredient.

In one or more embodiments, R¹ is selected from the group consisting of C₅-C₁₆ alkyl, C₅-C₁₆ alkenyl, and C₅-C₁₆ alkynyl; R₂ is selected from the group consisting of hydrogen, hydroxyl, —OC₁-C₆ alkyl, —OC₂-C₆ alkenyl, —OC₂-C₆ alkynyl, —OC₃-C₈ cycloalkyl; R₃ is selected from —CO₂H, —CO₂R₄ wherein R⁴ is C₁-C₆ alkyl, or an isosteric equivalent of a carboxy group; and Y is —(CH₂—CH₂)— or —(CH═CH)—. In some embodiments, R¹ is selected from the group consisting of C₅-C₁₆ alkenyl; and R² is selected from the group consisting of hydrogen or —OC₁-C₃ alkyl.

In one or more embodiments, the compound of Formula I is selected from the group consisting of 3-(4-farnesyloxyphenyl)-propionic acid, 3-(4-farnesyloxy-3-hydroxyphenyl)-propionic acid, 3-(4-farnesyloxy-3-methoxyphenyl)-propionic acid, ethyl esters thereof, and combinations of two or more thereof. In some embodiments, the compound of Formula I is comprises 3-(4-farnesyloxyphenyl)-propionic acid. In one or more embodiments, the concentration of the compound of Formula I is present in an amount ranging from about 0.00001% to 10%, by total weight of the composition. In some embodiments, the skincare composition comprises a botanical extract comprising the compound of Formula I. In one or more embodiments, the botanical extract comprises an extract of a plant of the genus Acronychia. In some embodiments, said botanical extract is an extract of Acronychia acidula. In one or more embodiments, said botanical extract is a polar extract. In some embodiments, the botanical extract is present in an amount ranging from about 0.00001% to about 5% by total weight of the composition. In one or more embodiments, the extract of Acronychia acidula comprises 3-(4-farnesyloxyphenyl)-propionic acid in a concentration ranging from about about 0.1% to about 30% by total weight of the extract. In some embodiments, the processed oat ingredient is selected from the group consisting of fermented oat, colloidal oat, oat extract, oat oil, and combinations thereof. In one or more embodiments, the composition further comprises an ingredient selected from the group consisting of surfactants, chelating agents, emollients, humectants, conditioners, preservatives, opacifiers, fragrances, and combinations of two or more thereof. In some embodiments, the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product. In one or more embodiments, the compound of Formula I is comprises 3-(4-farnesyloxyphenyl)-propionic acid, and the processed oat ingredient is selected from the group consisting of fermented oat, oat extract, colloidal oat and combinations thereof.

The embodiments described herein may be combined in any suitable way. For example, in some embodiments, the skincare composition comprises
a. 3-(4-farnesyloxyphenyl)-propionic acid; and
b. a processed oat ingredient selected from the group consisting of fermented oat, colloidal oat, oat extract, oat oil, and combinations thereof,
wherein the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

Another aspect of the invention pertains to a method for treating skin comprising topically applying any of the compositions described herein. In some embodiments, the method is a method for treating signs of aging, treating acne, smoothing skin texture, or brightening the skin. In one or more embodiments, the method is a method of increasing CRABP2 expression.

### DETAILED DESCRIPTION

It is believed that one skilled in the art can, based on the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

Unless otherwise indicated, percentages used to express amounts of ingredients are percentage by weight (referred to as “weight %,” “wt %”, “% by weight” or “% (W/W)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” includes the endpoints 4 and 9.

As used herein, a composition that is “essentially free” or “substantially free” of an ingredient means the composition that has about 2% or less of that ingredient by weight based on the total weight of the composition. Preferably, a composition that is essentially free of an ingredient has about 1% or less, more preferably about 0.5% or less, more preferably about 0.1% or less, more preferably about 0.05 or less, more preferably about 0.01% or less by weight based on the total weight of composition of the ingredient. In certain more preferred embodiments, a composition that is essentially free of an ingredient is free of the ingredient, i.e. has none of that ingredient in the composition.

As used herein, “cosmetically/dermatologically acceptable” means that the ingredients which the term describes are suitable for use in contact with tissues (e.g., the skin or hair) without undue toxicity, incompatibility, instability, irritation, allergic response, and the like. As will be recognized by one of skill in the art, cosmetically/dermatologically acceptable salts are acidic/anionic or basic/cationic salts.

As used herein, the term “safe and effective amount” means an amount of the compound, extract or of the composition sufficient to induce the desired effect, but low enough to avoid serious side effects. The safe and effective amount of the compound, extract, or composition will vary with e.g. the age, health and environmental exposure of the end user, the duration and nature of the treatment, the specific extract, ingredient, or composition employed, the particular pharmaceutically-acceptable carrier utilized, and like factors.

As used herein, the term “about” refers to within 5% weight, within 4% weight, within 3% weight, within 2.5% weight, within 2% weight, or within 1% weight of a disclosed value.

In general, IUPAC nomenclature rules are used herein and according to the following term definitions.

The term “substituted,” refers to a core molecule in which one or more hydrogen atoms have been replaced with that amount of substituents allowed by available valences. Substitution is not limited to the core molecule, but may also occur on a substituent radical, whereby the radical becomes a linking group.

The term “independently selected” refers to two or more substituents that may be selected from a substituent variable group, wherein the selected substituents may be the same or different.

The term “dependently selected” refers to one or more substituent variables that are specified in an indicated combination for substitution in a core molecule (e.g. variables that refer to groups of substituents appearing in a tabular list of compounds).

Acceptable salts from inorganic bases include, for example, sodium or potassium salts, and the like. Acceptable salts from organic bases include, for example, salts formed with primary, secondary, or tertiary amines, and the like.

One aspect of the invention pertains to a skincare composition comprising:
(a) a compound of Formula I:

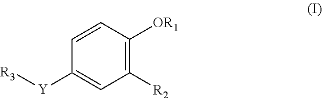

wherein:
   R₁ is selected from the group consisting of C₁-C₂₀ alkyl, C₂-C₂₀ alkenyl, C₂-C₂₀ alkynyl, and C₃-C₈ cycloalkyl or aryl;
   R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
   R₃ is selected from —CO₂H, —CO₂R₄ or an isosteric equivalent of a carboxy group, wherein R⁴ is C₁-C₆ alkyl, C₂-C₆ alkenyl, C₃-C₈ cycloalkyl or aryl; and
Y is —(CH₂—CH₂)—, —(CH═CH)—, or —(C≡C)—;
or a cosmetically acceptable salt thereof; and
(b) a processed oat ingredient. Such compositions have been shown to exhibit synergy in a variety of respects compared to the ingredients alone.

### Compound of Formula I

In one or more embodiments, R¹ is selected from the group consisting of C₅-C₁₆ alkyl, C₅-C₁₆ alkenyl, and C₅-C₁₆ alkynyl; R₂ is selected from the group consisting of hydrogen, hydroxyl, —OC₁-C₆ alkyl, —OC₂-C₆ alkenyl, —OC₂-C₆ alkynyl, —OC₃-C₈ cycloalkyl; R₃ is selected from —CO₂H, —CO₂R₄ wherein R⁴ is C₁-C₆ alkyl, or an isosteric equivalent of a carboxy group; and Y is —(CH₂—CH₂)— or —(CH═CH)— (or a cosmetically acceptable salt thereof). In some embodiments, R₁ is selected from the group consisting of C₅-C₁₆ alkenyl; and R² is selected from the group consisting of hydrogen or —OC₁-C₃ alkyl. In one or more embodiments, the compound of Formula I is selected from the group consisting of 3-(4-farnesyloxyphenyl)-propionic acid, 3-(4-farnesyloxy-3-hydroxyphenyl)-propionic acid, 3-(4-farnesyloxy-3-methoxyphenyl)-propionic acid, ethyl esters thereof, and combinations of two or more thereof. In one or more embodiments, the compound of the above Formula I is 3-(4-farnesyloxyphenyl)-propionic acid and/or its ethyl ester. In preferred embodiments, the compound of Formula I is comprises 3-(4-farnesyloxyphenyl)-propionic acid. The 3-(4-farnesyloxyphenyl)-propionic acid and/or its ethyl ester can be synthesized using conventional organic synthesis processes.

The compound of Formula I may be present in an amount ranging from about 0.00001% to 10%, or about 0.0001 to about 10%, or about 0.001 to about 5%, or about 0.001% to about 1%, or about 0.01% to about 3%, about 0.01% to about 1%, about 0.01% to about 0.5%, or about 0.005% to about 1.5%, or about 0.005% to about 0.06%, or about 0.009% to about 0.06%, or about 0.009% to about 0.03% by total weight of the composition.

Compounds according to Formula I can also be obtained from natural sources. For example, a compound according to Formula I may be found in a botanical extract. Accordingly, the composition may comprise a botanical extract comprising the compound of Formula I.

In one or more embodiments, the botanical extract is an extract of a plant of the genus Acronychia. In further embodiments, the botanical extract is an extract of Acronychia acidula (also known as lemon aspen). In one or more embodiments, at least one compound of the above Formula I is present in the extract of Acronychia at a concentration equal to or greater than about 0.01 to about 30%, or about 0.1% to about 30%, or about 0.1 to about 20%, or about 1% to about 20%, or about 1% to about 10%, or about 1.5% to about 9%, or about 3% to about 9%, by weight of the Acronychia extract. In further embodiments, the extract of Acronychia acidula comprises 3-(4-farnesyloxyphenyl)-propionic acid in a concentration ranging from about 1% to about 10% by total weight of the extract.

Suitable extracts may be obtained using conventional methods including, but not limited to, direct extraction of material from the biomass by grinding, macerating, pressing, squeezing, mashing, centrifuging, and/or processes such as cold percolation, agitation/distillation, microwave assisted extraction, supercritical/subcritical CO₂ compressed gas extraction with or without polar modifiers, pressurized solvent extraction, accelerated solvent extraction, pressurized or normal hot water extraction, surfactant assisted pressurized hot water extraction, oil extraction, membrane extraction, Soxhlet extraction, the gold finger distillation/extraction and/or processes disclosed, for example, in U.S. Pat. Nos. 7,442,391, 7,473,435, and 7,537,791 to Integrated Botanical Technologies, LLC, incorporated herein by reference, and the like, or by other methods such as solvent extraction, and the like.

Any of a variety of solvents including polar solvents, non-polar solvents, or combinations of two or more thereof may be used in methods comprising solvent extraction. Suitable polar solvents include polar inorganic solvents such as water and the like, polar organic solvents such as alcohols and corresponding organic acids, for example C₁-C₈ alcohols including methanol, ethanol, propanol, butanol, and the like and organic acids, including acetic acid, formic acid, propanoic acid, and the like, polyols and glycols, including C₁-C₈ polyols/glycols and the like, and combinations of two or more thereof. Suitable non-polar solvents include non-polar organic solvents such as alkanes, including C₁-C₈ alkanes, cycloalkanes, including C₁-C₈ alkanes, alkyl ethers, including C₁-C₈ alkyl ethers, Petroleum ethers, ketones, including C₁-C₈ ketones, methylene chloride, ethyl acetate, xylene, toluene, chloroform, vegetable oil, mineral oil and the like. In another embodiment extraction may be obtained by non-polar solvents described above or supercritical fluid extraction with or without a polar modifier such as C₁-C₈ alcohols, water, C₁-C₈ polyols/glycols or C₁-C₈ organic acids.

In one or more embodiments, the extract comprises an extract of Acronychia acidula. In some embodiments, the extract of the invention comprises a combination of polar and non-polar extracts of Acronychia acidula fruit. In another embodiment, the extract of the invention comprises alcoholic or glycolic extracts of Acronychia acidula fruit.

In one or more embodiments, the extract is a polar extract. In further embodiments, the extract is a polar extract prepared using a polar solvent comprising water, C₁-C₈ alcohols, C₁-C₈ polyols, or C₁-C₈ glycols, or combinations of two or more thereof. In certain embodiments, the extract is extracted using one or more C₁-C₄ alcohols, C₁-C₄ polyols, and/or C₁-C₄ glycols. In one or more embodiments, the extract is prepared using a solvent comprising methanol, ethanol, or a combination thereof with or without presence of water. In further embodiments, the extract is a polar extract extracted from Acronychia acidula fruit using a combination of alcohol and water.

In one or more embodiments, the extract is a non-polar extract prepared using a non-polar solvent comprising one or more C₁-C₈ alkanes, C₁-C₈ cycloalkanes, C₁-C₈alkyl ethers, C₁-C₈ alkyl esters and/or chloroform, more preferably one or more C₁-C₈ alkanes, C₁-C₈ alkyl esters and/or chloroform. In further embodiments, extract is a non-polar extract prepared using hexanes, ethyl acetate, chloroform, or mixtures of two or more thereof. In yet further embodiments, the extract is a non-polar extract prepared using ethyl acetate.

For example, an extract using the fruit of Acronychia acidula may be prepared by homogenizing the fruit in a blender for 30 seconds with denatured alcohol in equal part to fruit. The pulp can then mixed and stirred for another 24 hours at ambient temperature (22 to 26 degrees C.). Additional denatured alcohol may be added as needed to keep the pulp covered well in alcohol. The mixture then can then be gravity filtered, and the resulting filter cake washed with additional amounts of denatured alcohol. The total filtrate then may then be dried under reduced pressure to remove alcohol. The residue can then be freeze dried to obtain dry matter free of extraction solvent and water. The extraction may be repeated on the filter cake a few times with an extract yield of 5-7% regularly obtained from each extraction.

Another example of the preparation of Acronychia acidula fruit extract is as follows: 500 μm of freeze-dried fruits of Acronychia acidula may be sliced into approximately 5 mm cubes and soaked with 5 L of ethanol at a ratio of 1:10 (raw material to solvent) and stirred at room temperature for 12 hours. The suspension may then be filtered and resulting filtrate concentrated under low pressure to afford a concentrate. The concentrate can then further be dried by freeze-drying methods to obtain 325 μm of residual material called crude extract (65% yield). A sample of the crude extract, 200 μm, can then be taken up in 1 L ethanol and stirred at room temperature overnight. The mixture may then be filtered and dried at reduced pressure and at low temperature The extract may be present in an amount of about 0.00001, 0.0001, 0.001, 0.01, 0.1, 0.2, 0.3, 0.4, 0.5, 0.75, 1, 1.5 or 2 to about 0.00005, 0.0005, 0.005, 0.05, 0.5, 1, 1.5, 2, 2.5, 3, 4 or 5 wt. % by total weight of the composition. In one or more embodiments, the extract is present in an amount of about 0.01 to about 5 wt. % by total weight of the composition. In further embodiments, the extract is present in an amount of about 0.01 to about 3 wt. % by total weight of the composition. In one or more embodiments, the extract is present in an amount of about 0.1 to about 3 wt. % by total weight of the composition. In further embodiments, the extract is present in an amount of about 0.2 to about 2.5 wt. % by total weight of the composition. In one or more embodiments, the extract is present in an amount of about 0.5 to about 2 wt. % by total weight of the composition. In further embodiments, the extract is present in an amount of about 0.5 to about 1.5 wt. % by total weight of the composition. In one or more embodiments, the extract is present in an amount of about 0.1 to about 3 wt. % by total weight of the composition. In further embodiments, the extract is present in an amount of about 0.2 to about 1 wt. % by total weight of the composition. In one or more embodiments, the extract is present in an amount of about 0.25 to about 0.5 wt. % by total weight of the composition. In further embodiments, the extract is present in an amount of about 0.1 to about 1 wt. % by total weight of the composition.

### Processed Oat Ingredient

As used herein, the term “processed oat ingredient” refers to an ingredient that is typically derived from a part of the oat plant (Avena sativa). Said ingredient can be either a processing (e.g., an extract, milling, fermenting) of one or more parts of the oat plant (e.g., grain, leaf, stem, seed) (e.g., an extract) or can be a molecule found in the oat plant (e.g., beta-glucan, flavonoids, avenanthramides, lipids, peptides, etc.). The definition is intended to cover processed oat ingredients which are derived from sources other than oat (e.g., from another plant or chemically synthesized), but are otherwise associated with oat. In one or more embodiments, the processed oat ingredient is selected from the group consisting of oat extract, colloidal oatmeal (used interchangeably with oat flour), oat bran, oat protein, oat peptide, oat oil, fermented oat, avenanthramides, beta-glucan, modified oat grain material (e.g., chemically, enzymatically-, microorganism-modified), and combinations thereof. As used herein, “colloidal oatmeal” means the powder resulting from the grinding and further processing of whole oat grain meeting United States Standards for Number 1 or Number 2 oats. The colloidal oatmeal has a particle size distribution as follows: not more than 3 percent of the total particles exceed 150 micrometers in size and not more than 20 percent of the total particles exceed 75 micrometers in size. Examples of suitable colloidal oatmeals include, but are not limited to, “Tech-0” available from the Beacon Corporation and colloidal oatmeals available from Quaker. In one or more embodiments, the processed oat ingredient comprises oat extract, colloidal oatmeal, and oat oil. In some embodiments, the processed oat ingredient comprises oat extract. In one or more embodiments, the processed oat ingredient comprises colloidal oatmeal. In some embodiments, the processed oat ingredient comprises oat oil. In some embodiments, the processed oat ingredient is selected from the group consisting of oat extract, colloidal oatmeal, oat oil and combinations thereof. In some embodiments, the processed oat ingredient comprises oat extract, colloidal oatmeal, and oat oil. In one or more embodiments, the processed oat ingredient comprises avenanthramides. In some embodiments, the processed oat ingredient comprises fermented oat. In one or more embodiments, the processed oat ingredient comprises beta-glucan. In some embodiments, the oat extract is free or substantially free of protein.

In further embodiments, the processed oat ingredient is selected from the group consisting of fermented oat, colloidal oat, oat extract, oat oil, and combinations thereof. In yet further embodiments, the processed oat ingredient is selected from the group consisting of fermented oat, colloidal oat, oat extract, and combinations thereof.

The processed oat ingredient may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, 10, 11, 12, 13, 14 or 15 wt %. In one or more embodiments, the processed oat ingredient may be present in amounts ranging from about 0.001 to about 10 wt %. In further embodiments, the processed oat ingredient may be present in amounts ranging from about 0.01 to about 8 wt %. In one or more embodiments, the processed oat ingredient may be present in amounts ranging from about 0.1 to about 6 wt %. In further embodiments, the processed oat ingredient may be present in amounts ranging from about 0.2 to about 5 wt %.

In embodiments where the processed oat ingredient comprises colloidal oat, the colloidal oat may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, 10, 11, 12, 13, 14 or 15 wt %. In one or more embodiments, the colloidal oat may be present in amounts ranging from about 0.001 to about 10 wt %. In further embodiments, the colloidal oat may be present in amounts ranging from about 0.01 to about 8 wt %. In one or more embodiments, the colloidal oat may be present in amounts ranging from about 0.1 to about 5 wt %. In further embodiments, the colloidal oat may be present in amounts ranging from about 0.5 to about 4 wt %. In one or more embodiments, the colloidal oat may be present in amounts ranging from about 0.5 to about 2 wt %, or about 1% wt %. In further embodiments, the colloidal oat may be present in amounts ranging from about 2 to about 4 wt %, or about 3 wt %.

In embodiments where the processed oat ingredient comprises fermented oat, the fermented oat may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, 10, 11, 12, 13, 14 or 15 wt %. In one or more embodiments, the fermented oat may be present in amounts ranging from about 0.001 to about 10 wt %. In further embodiments, the fermented oat may be present in amounts ranging from about 0.01 to about 8 wt %. In one or more embodiments, the fermented oat may be present in amounts ranging from about 0.1 to about 6 wt %. In further embodiments, the fermented oat may be present in amounts ranging from about 0.5 to about 5 wt %.

In embodiments where the processed oat ingredient comprises oat extract, the oat extract may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, 10, 11, 12, 13, 14 or 15 wt %. In one or more embodiments, the oat extract may be present in amounts ranging from about 0.001 to about 10 wt %. In further embodiments, the oat extract may be present in amounts ranging from about 0.01 to about 8 wt %. In one or more embodiments, the oat extract may be present in amounts ranging from about 0.1 to about 5 wt %. In further embodiments, the oat extract may be present in amounts ranging from about 0.5 to about 4 wt %.

### Methods

Another aspect of the invention pertains to a method for treating skin, the method comprising topically applying to skin a composition in accordance with one more embodiments of the invention. As used herein, “treatment” or “treating” means the amelioration, prophylaxis, or reversal of a condition, disease, or disorder, or at least one discernible symptom thereof. In one embodiment, “treatment” or “treating” refers to an amelioration, prophylaxis, or reversal of at least one measurable physical parameter related to the condition, disease, or disorder being treated, not necessarily discernible in or by the subject being treated. In another embodiment, “treatment” or “treating” refers to inhibiting or slowing the progression of a condition, disease, or disorder, either physically, e.g., stabilization of a discernible symptom, physiologically, e.g., stabilization of a physical parameter, or both. In another embodiment, “treatment” or “treating” refers to delaying the onset of a condition, disease, or disorder.

The compositions/compounds described herein may be used in treating skin for treating acne, treating the signs of aging (e.g., wrinkles), improving skin barrier function and/or lightening skin. In one or more embodiments, said treatment is for a subject who has a condition or a history of a condition selected from the group consisting of atopic dermatitis, rosacea, seborrheic dermatitis, psoriasis, dry skin, flaky skin.

Compositions of the invention are suitable for improving the texture of skin or improving the firmness of skin, or any of the conditions/symptoms described below.

As used herein, “improving the texture of skin” means the smoothing of the surface of the skin to remove either bumps or crevasses on the skin surface.

As used herein, “improving the firmness of skin” means the enhancing of the firmness or elasticity of the skin, preventing the loss of firmness or elasticity of skin, or preventing or treating sagging, lax and loose skin.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

As used herein, “age spots” means a condition of the skin associated with discrete pigmentation, e.g., small areas of darker pigmentation that may develop on the face as well as the hands.

Signs of skin aging also include the presence of diminished skin thickness, and abnormal or diminished synthesis of collagen, glycosaminoglycans, proteoglycans, elastin, or glycoproteins including fibronectin. In one embodiment, the sign of aging is selected from the abnormal or diminished synthesis of collagen, glycosaminoglycans, proteoglycans, elastin, or glycoproteins including fibronectin. In another embodiment, the sign of skin aging is diminished synthesis of collagen or elastin.

Examples of skin aging that may be treated by topical use of the compositions of this invention include, but are not limited to, wrinkles on the skin. As used herein, the term “wrinkle” includes fine line, fine wrinkles, coarse wrinkles, cellulite, scars, and stretch marks. Examples of wrinkles include, but are not limited to, fine lines around the eyes (e.g., “crow's feet”), forehead and cheek wrinkles, frown-lines, and laugh-lines around the mouth.

As used herein, “topical use” and “topically applying” means directly laying on or spreading on the skin, hair, or nail, e.g., by use of the hands or an applicator such as a wipe.

The compositions are also suitable for treating or preventing acne. As used herein, “acne” refers to disorders resulting from the actions of hormones and other substances on the sebaceous glands and hair follicles, typically leading to clogged pores and the formation of inflammatory or non-inflammatory lesions on the skin. Specifically, it relates to blemishes, lesions, or pimples, pre-emergent pimples, blackheads, and/or whiteheads. As used herein, a “pre-emergent pimple” is an inflamed follicle that is not visually apparent on the surface of the skin with the naked eye (e.g., as a lesion).

The compositions of the invention are also suitable for treating or preventing rosacea. As used herein, “rosacea” means skin with persistent erythema with or without papules, pustules, or nodules.

The compositions of the invention are also suitable for reducing epidermal hyperkeratinzation. Accordingly, the composition may be used for treatment or prevention of conditions characterized by hyperkeratinzation, such as acne or warts.

In one or more embodiments, one or more of the methods described herein modify the expression of one or more biomarkers. For example, in one or more embodiments, the method may be a method of increasing CRABP2, HAS2, or HBEGF expression in skin.

The embodiments described herein may be combined in any suitable combination. For example, an exemplary embodiment pertains to a method of treating acne, signs of aging and/or lightening skin, the method comprising applying to the skin a composition comprising:
(a) an extract of Acronychia acidula and/or 3-(4-farnesyloxyphenyl)-propionic acid; and
(b) a processed oat ingredient selected from the group consisting of fermented oat, colloidal oat, oat extract, oat oil, and combinations thereof,
wherein the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product. In further embodiments, the processed oat ingredient is selected from fermented oat, colloidal oat, oat extract, and combinations thereof. In yet further embodiments, the processed oat is selected from fermented oat, colloidal oat and combinations thereof. The 3-(4-farnesyloxyphenyl)-propionic acid may be chemically synthesized, naturally occurring in a botanical extract, or chemically synthesized and added to a botanical extract. In one or more embodiments, the applying step comprises applying to the skin a composition comprising a botanical extract is an extract of a plant of the genus Acronychia. In further embodiments, the botanical extract is an extract of Acronychia acidula. In yet further embodiments, the extract is a polar extract. In one or more embodiments, the extract is present in an amount of about 0.1 to about 3 wt. % by total weight of the composition. In further embodiments, the extract is present in an amount of about 0.4 to about 1.5 wt. % by total weight of the composition.

The compositions described herein may be applied to any skin in need of treatment on the human body. For example, application may be made to any one or more of the skin of the face, neck, chest, back, arms, axilla, hands and/or legs. In certain preferred embodiments, the method comprises applying a composition according to one or more embodiments of the invention to skin of the face.

Any suitable method of applying the extract to the skin in need may be used in accordance with the present invention. For example, the extract may be applied directly from a package to the skin in need, by hand to the skin in need, or may be transferred from a substrate such as a wipe or mask, or a combination of two or more thereof. In other embodiments, the extract may be applied via a dropper, tube, roller, spray, patch or added to a bath or otherwise to water to be applied to the skin, and the like.

In one or more embodiments, the methods of the present invention further comprise the step of leaving the composition in contact with the skin for period of time. For example, in certain preferred embodiments after application, the compound is left in contact with the skin for a period of about 15 minutes or greater. In certain more preferred embodiments, the extract is left in contact with the skin for about 20 minutes or greater, more preferably about 1 hour or greater.

In some embodiments, the method of the present invention comprises a regimen comprising applying the composition to skin multiple times over a selected period of time. For example, in certain embodiments, the present invention provides a method of treating signs of aging comprising applying to skin in need of antiaging a composition in accordance with one or more embodiments of the invention once or twice daily for at least 12 weeks, preferably at least 8 weeks and more preferably for at least 2 weeks.

### Compositions

Any suitable carrier may be used in the compositions of the present invention. Preferably, for a skin care composition, the carrier is a cosmetically-acceptable carrier. As will be recognized by those of skill in the art, cosmetically-acceptable carriers comprise carriers that are suitable for use in contact with the body, in particular the skin for antiaging applications, without undue toxicity, incompatibility, instability, irritation, allergic response, and the like. A safe and effective amount of carrier is from about 50% to about 99.999%, preferably from about 80% to about 99.9%, more preferably from about 99.9% to about 95%, most preferably from about 99.8% to about 98% of the composition. The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cP to about 200,000 cP. Examples of suitable cosmetically-acceptable carriers include cosmetically-acceptable solvents and materials for cosmetic solutions, suspensions, lotions, creams, serums, essences, gels, toners, sticks, sprays, ointments, liquid washes and soap bars, shampoos, hair conditioners, pastes, foams, mousses, powders, shaving creams, wipes, patches, strips, powered patches, microneedle patches, bandages, hydrogels, film-forming products, facial and skin masks, makeup, liquid drops, and the like. These product types may contain several types of cosmetically-acceptable carriers including, but not limited to solutions, suspensions, emulsions such as microemulsions and nanoemulsions, gels, solids, liposomes, other encapsulation technologies and the like. In one or more embodiments, the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

The following are non-limitative examples of such carriers. Other carriers can be formulated by those of ordinary skill in the art. In one embodiment, the carrier contains water. In a further embodiment, the carrier may also contain one or more aqueous or organic solvents. Examples of organic solvents include, but are not limited to: dimethyl isosorbide; isopropylmyristate; surfactants of cationic, anionic and nonionic nature; vegetable oils; mineral oils; waxes; gums; synthetic and natural gelling agents; alkanols; glycols; and polyols. Examples of glycols include, but are not limited to, glycerin, propylene glycol, butylene glycol, pentylene glycol, hexylene glycol, polyethylene glycol, polypropylene glycol, diethylene glycol, triethylene glycol, capryl glycol, glycerol, butanediol and hexanetriol, and copolymers or mixtures thereof. Examples of alkanols include, but are not limited to, those having from about 2 carbon atoms to about 12 carbon atoms (e.g., from about 2 carbon atoms to about 4 carbon atoms), such as isopropanol and ethanol. Examples of polyols include, but are not limited to, those having from about 2 carbon atoms to about 15 carbon atoms (e.g., from about 2 carbon atoms to about 10 carbon atoms) such as propylene glycol. The organic solvents may be present in the carrier in an amount, based upon the total weight of the carrier, of from about 1 percent to about 99.99 percent (e.g., from about 20 percent to about 50 percent). Water may be present in the carrier (prior to use) in an amount, based upon the total weight of the carrier, of from about 5 percent to about 95 percent (e.g., from about 50 percent to about 90 percent). Solutions may contain any suitable amounts of solvent, including from about 40 to about 99.99%. Certain preferred solutions contain from about 50 to about 99.9%, from about 60 to about 99%, from about 70 to about 99%, from about 80 to about 99%, or from about 90 to 99%.

A lotion can be made from such a solution. Lotions typically contain at least one emollient in addition to a solvent. Lotions may comprise from about 1% to about 20% (e.g., from about 5% to about 10%) of an emollient(s) and from about 50% to about 90% (e.g., from about 60% to about 80%) of water. As used herein, “emollients” refer to materials used for the prevention or relief of dryness, as well as for the protection of the skin or hair. Examples of emollients include, but are not limited to, those set forth in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (hereinafter “ICI Handbook”).

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid hydrocarbons. An ointment may contain from about 2% to about 10% of an emollient(s) plus from about 0.1% to about 2% of a thickening agent(s).

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 1673-1686.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, or wipe). The composition of the present invention can also be combined with a solid, semi-solid or dissolvable substrate (eg., a wipe, mask, pad, glove or strip).

The compositions of the present invention may further comprise any of a variety of additional cosmetically active agents, although they are preferably formulated to account for use on skin. Examples of suitable additional active agents include: additional skin lightening agents, darkening agents, anti-acne agents, shine control agents, antimicrobial agents such as anti-yeast agents, anti-fungal, and anti-bacterial agents, anti-inflammatory agents, anti-parasite agents, external analgesics, sunscreens, photo-protectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, fluorides, teeth whitening agents, anti-plaque agents, and plaque-dissolving agents, odor-control agents such as odor masking or pH-changing agents, and the like. Examples of various suitable additional cosmetically acceptable actives include hydroxy acids, benzoyl peroxide, D-panthenol, UV filters such as but not limited to avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, carotenoids, free radical scavengers, spin traps, retinoids and retinoid precursors such as retinol, retinoic acid and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, amino acids such a proline, vitamins, lactobionic acid, acetyl-coenzyme A, niacin, riboflavin, thiamin, ribose, electron transporters such as NADH and FADH2, and other botanical extracts such as oat, aloe vera, Feverfew, Soy, Shiitake mushroom extracts, and derivatives and mixtures thereof.

In one or more embodiments, the compositions of the present invention are skin care compositions that comprise a compound of Formula I and at least one skin lightening active agent. Examples of suitable skin lightening active agents include, but are not limited to, tyrosinase inhibitors, melanin-inhibiting agents, melanosome transfer inhibiting agents including PAR-2 antagonists, exfoliants, sunscreens, retinoids, antioxidants, Tranexamic acid, skin bleaching agents, allantoin, opacifiers, talcs and silicas, zinc salts, and the like, and other agents as described in Solano et al. Pigment Cell Res. 2006, 19 (550-571). Examples of suitable tyrosinase inhibitors include but, are not limited to, Vitamin C and its derivatives, Vitamin E and its derivatives, Kojic Acid, Arbutin, resorcinols, hydroquinone, Flavones e.g. Licorice flavanoids, Licorice root extract, Mulberry root extract, Dioscorea Coposita root extract, Saxifraga extract and the like, Ellagic acid, Salicylates and derivatives, Glucosamine and derivatives, Fullerene, Hinokitiol, Dioic acid, Acetyl glucosamine, Magnolignane, combinations of two or more thereof, and the like. Examples of Vitamin C derivatives include, but are not limited to, ascorbic acid and salts, Ascorbic Acid-2-Glucoside, sodium ascorbyl phosphate, magnesium ascorbyl phosphate, and natural extract enriched in Vitamin C. Examples of Vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in Vitamin E derivatives. Examples of resorcinol derivatives include, but are not limited to, resorcinol, 4-substituted resorcinols like 4alkylresorcinols such as 4-butyresorcinol (rucinol), 4-hexylresorcinol, phenylethyl resorcinol, 1(2,4-dihydroxyphenyl)-3-(2,4-dimethoxy-3-methylphenyl)-Propane and the like and natural extracts enriched in resorcinols. Examples of salicylates include, but are not limited to, salicylic acid, acetylsalicylic acid, 4-methoxysalicylic acid and their salts. In certain preferred embodiments, the tyrosinase inhibitors include a 4-substituted resorcinol, a Vitamin C derivative, or a Vitamin E derivative. In more preferred embodiments, the tyrosinase inhibitor comprises Phenylethyl resorcinol, 4-hexyl resorcinol, or ascorbyl-2-glucoside.

Examples of suitable melanin-degradation agents include, but are not limited to, peroxides and enzymes such as peroxidases and ligninases. In certain preferred embodiments, the melanin-inhibiting agents include a peroxide or a ligninase.

Examples of suitable melanosome transfer inhibiting agents including PAR-2 antagonists such as soy trypsin inhibitor or Bowman-Birk Inhibitor, Vitamin B3 and derivatives such as Niacinamide, Essential soy, Whole Soy, Soy extract. In certain preferred embodiments, the melanosome transfer inhibiting agents includes a soy extract or niacinamide.

Examples of exfoliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfoliation such as microdermabrasion. In certain preferred embodiments, the exfoliant include glycolic acid or salicylic acid.

Examples of sunscreens include, but are not limited to, avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, and the like.

Examples of retinoids include, but are not limited to, retinol, retinaldehyde, retinoic acid, retinyl palmitate, tretinoin, tazarotene, bexarotene and Adapalene. In certain preferred embodiments, the retinoid is retinol. Nevertheless, in one or more embodiments, the composition is essentially free of retinoids or retinoid precursors; and in further embodiments, is free of retinoids or retinoid precursors.

Examples of antioxidants include, but are not limited to, water-soluble antioxidants such as sulfhydryl compounds and their derivatives (e.g., sodium metabisulfite and N-acetyl-cysteine, glutathione), lipoic acid and dihydrolipoic acid, stilbenoids such as resveratrol and derivatives, lactoferrin, and ascorbic acid and ascorbic acid derivatives (e.g., ascobyl-2-glucoside, ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, feverfew, parthenolide-free feverfew, oat extracts, pomelo extract, wheat germ extract, Hesperidin, Grape extract, Portulaca extract, Licochalcone, chalcone, 2,2′-dihydroxy chalcone, Primula extract, propolis, and the like.

The additional cosmetically active agent may be present in a composition in any suitable amount, for example, in an amount of from about 0.0001% to about 20% by weight of the composition, e.g., about 0.001% to about 10% such as about 0.01% to about 5%. In certain preferred embodiments, in an amount of 0.1% to 5% and in other preferred embodiments from 1% to 2%.

A variety of other materials may also be present in the compositions of the present invention. These include, for example, chelating agents, humectants, opacifiers, conditioners, preservatives, fragrances and the like. The compositions may include surfactants, for example, those selected from the group consisting of anionic, non-ionics, amphoteric, cationic, or a combination of two or more thereof.

The compositions of the present invention may also contain chelating agents (e.g., EDTA) and preservatives (e.g., parabens). Examples of suitable preservatives and chelating agents are listed in pp. 1626 and 1654-55 of the ICI Handbook. In addition, the compositions useful herein can contain conventional cosmetic adjuvants, such as colorants such as dyes and pigments, opacifiers (e.g., titanium dioxide), and fragrances.

In certain preferred embodiments, the present invention comprises applying a compound or composition of the invention via a substrate comprising such material. Any suitable substrate may be used in the present invention. Examples of suitable substrates and substrate materials are disclosed, for example, in U.S. Published Application Nos. 2005/022683 and 2009/0241242 which are incorporated herein by reference in their entirety. In certain preferred embodiments, the substrate is a wipe or a facial mask.

The composition and products containing such compositions of this invention may be prepared using methodology that is well known by an artisan of ordinary skill.

### EXAMPLES

The below examples evaluate various samples including processed oat ingredients, Lemon aspen extract and combinations thereof. Some examples pertain to evaluation of the of retinol-responsive genes such as RARg, CRABP2, HbEGF, HAS2, and HAS3. The upregulation of these genes is associated with skin benefits provided by retinoids (e.g., anti-aging effects, reduction of wrinkles, acne, and tone benefits. Some examples pertain to evaluation of the gene for IL-8, an inflammatory mediator interleukin. Expression of the IL-8 gene is associated with increased inflammation.

### Example 1: Retinoic Acid Receptor-Gamma (RARg) Transactivation Assay of Lemon Aspen Extract Alone and in Combination with Colloidal Oat

RARg agonism of several samples was assessed using the GENEBLAZER RAR gamma-UAS-bla HEK 293T cell-based assay (Thermofisher, Waltham MA), in which activation of RARg results in transcription of beta lactamase. FRET is observed when a fluorescence resonance energy transfer (FRET) probe is added to cells. However, upon receptor activation and subsequent beta lactamase expression, the probe is cleaved and FRET is no longer observed. HEK293T cells in culture were washed with phosphate buffered saline (PBS), trypsinized, resuspended in growth media consisting of DMEM with GlutaMAX (Invitrogen Waltham, MA), 10% Fetal Bovine Serum (FBS), 0.1 mM non-essential amino acid (NEAA), 25 mM HEPES, 1× Penn/Strep (100 units Penn/100 μg/mL Strep), 80 μg/mL Hygromycin, 80 μg/mL Zeocin, and then counted using a cell counter, Vi-Cell (Breckman Coulter, Brea, CA). The cells were then spun down and resuspended in assay buffer consisting of Phenol red-free DMEM (Invitrogen), 2% Charcoal-stripped FBS, 0.1 mM NEAA, 1 mM Sodium pyruvate, 1× Penn/Strep (at 0.5E6 cells/mL). 15 k cells in 30 μL assay buffer were plated per well in Greiner black walled, clear flat bottom plates using a combi dispenser. The last two columns were filled with assay buffer. Lemon aspen extract, 30% stock solution comprising 45% Polysorbate 20, 21.5% water, and 3.5% butylated hydroxytoluene (Southern Cross Botanicals, Knockrow, NSW, Australia) was diluted 1:30 in media to make 1% lemon aspen extract in solution. The 1% lemon aspen extract solution was further diluted in media to make concentrations of (5×10⁻⁵) %, (1.5×10⁻⁴) %, (4.5×10⁻⁴) %, (1.4×10⁻³) %, (4.15×10⁻³) %, and (1.25×10⁻²) % lemon aspen extract. Acronychia acidula Fruit extract used in the examples typically contains between about 1% and 10% 3-(4-farnesyloxyphenyl)-propionic acid, with these samples containing 1.11% 3-(4-farnesyloxyphenyl)-propionic acid) by weight of the extract in the extract in carrier. Colloidal oat (Avena sativa Kernel Flour, Oat Cosmetics, Southampton, United Kingdom), was dissolved 100 mg in 1.0 mL media for a concentration of 10% Colloidal oat 2 hours before use, then the 10% Colloidal oat solution was sonicated for 10 minutes and centrifuged for 5 minutes. The Colloidal oat solution was further diluted to prepare a 1.65% Colloidal oat solution. 10 μL of test samples were added to the cells achieving the final concentrations listed in Table 1. Cells were spun down at 800 rpm for 2 min without brakes, and left at room temperature for 15 min prior to overnight incubation at 37° C.

After 18-20 h of incubation, 8 μL FRET dye (Invitrogen, K1096) were added using a liquid dispenser sold under the tradename TEMPEST dispenser (FORMULATRIX, Bedford, MA, after which plates were spun down at 800 rpm for 2 min without brakes and incubated in the dark at room temperature for 2 h. The bottoms of the plates were cleaned using an anti-static cleaning wipe before measuring fluorescence using BMG PHERSTAR (Cary, NC) plate reader (using FRET module, 10 reads, gain for both channels=1000, Ex 409, Em1 460, Em2 530). Average Em1 and Em2 values from assay buffer only wells were subtracted from the test Em1 and Em2 values respectively, and subsequent determination of Em1/Em2 ratio represented RARg activity. Dimethyl sulfoxide (DMSO) and All-trans Retinoic Acid (ATRA) were used as neutral and stimulator control respectively for normalization.

Combinations of Colloidal oat and Lemon aspen extract were tested for RARg activity in Dose Response format for Lemon aspen extract. In the presence of lemon aspen extract, Colloidal oat significantly augmented RARg activity. Results are shown below in Table 1 and graphically in FIG. 1. The results surprisingly showed colloidal oat augmented Lemon aspen extract's retinoid bioactivity. This result is surprising given the fact that oat is not generally known for activation of the RARg gene.

TABLE 1
Dose response of Lemon aspen extract for RARg activity

| | RARg Activity | Lemon aspen extract + 0.33% |
|---|---|---|
| | Lemon aspen | Colloidal oat - RARg |
| Lemon Aspen (%) | extract only | Activity |
| (1 × 10⁻⁵)% | 4 | 1 |
| (3 × 10⁻⁵)% | 10 | 7 |
| (9 × 10⁻⁵)% | 30 | 31 |
| (2.8 × 10⁻⁴)% | 48 | 57 |
| (8.3 × 10⁻⁴)% | 44 | 68 |
| (2.5 × 10⁻³)% | 23 | 47 |

### Example 2: Bioactivity Assay of CRAPB2, IL8 and HAS2 in Human Dermal Fibroblasts of Fermented Oat and Lemon Aspen Extract, Alone and in Combination

CRAPB2, IL8 and HAS2 were evaluated in several samples containing fermented oat, lemon aspen extract, and combinations of the two in varying amounts. Lemon aspen extract, 30% stock solution (as above, Southern Cross Botanicals, Knockrow, NSW, Australia) was diluted in DMEM (Dulbecco's Modified Eagle Medium) with GlutaMAX media supplemented with 1% FBS (Fetal Bovine Serum) and 1× Penn/Strep to (6×10⁻⁴) % and (6×10⁻⁵) % lemon aspen extract. Fermented Oat (aurafirm P, Oat Cosmetics, Hampshire, United Kingdom) was diluted in media to provide 0.2% and 1.0% Fermented Oat solutions. 100 μL of each test solution were added to each well to achieve the final concentrations as listed in Tables 2-4 below.

Primary human dermal fibroblasts were acquired from Lifeline Cell Technology, LLC (Frederick, MD 21701; lot 0967). The donor demographics were as follows: 18 year old, Female, Caucasian, fibroblasts isolated from breast. Fibroblasts were cultured and expanded using Dermal Fibroblast Culture Medium (Zenbio, Inc, Durham, NC 27713). In preparation for the experiment, fibroblasts were seeded at a concentration of 10,000 cells/well in 96-well plates. Cells were treated 6-24 hours upon seeding with DMEM with GlutaMAX media supplemented with 1% FBS and 1× Penn/Strep. Upon 30 hours of treatment, a Cell-to-CT kit (Invitrogen, Waltham, MA) was used to isolate RNA per manufacturer's instructions and converted to cDNA. The cDNA was evaluated by qPCR (using the QuantStudio™ 7 Flex Real-Time PCR System from Applied Biosystems, Waltham, MA). Gene expression assays sold under the tradename TAQMAN (ThermoFisher Scientific, Bridgewater, NJ) for cellular retinoic acid binding protein 2 (CRABP2), interleukin-8 (IL8), and polymerase (RNA) II polypeptide A (POLR2A), were used. The expression of these genes was normalized against the expression of the human POLR2A housekeeping gene. The fold changes were calculated in comparison to the untreated control and two-tailed two-sample Student t-tests (Microsoft Office Excel 2007; Microsoft, Redmond, WA, USA) were performed. The calculation of synergy was performed as follows: Briefly, the resultant fold change over untreated of each ingredient alone was added together, and a 1-fold change was subtracted to account for untreated contributions. If the result was smaller than the resultant bioactivity of the ingredient combination, then it was considered synergistic, since the result was greater than the sum of its parts. Results are shown as Average Fold Change over Untreated in Tables 2-4.

TABLE 2
CRABP2 Average Fold Change over Untreated

| | | Average | | |
|---|---|---|---|---|
| | | Fold Change | | |
| | | over | Standard | Synergy |
| Sample No. | Treatment | untreated | Deviation | Comparison |
| 2-1 (Comp.) | Untreated | 1.00 | 0.12 | |
| 2-2 (Comp.) | (3 × 10⁻⁵)% Lemon aspen | 1.13 | 0.19 | |
| | extract | | | |
| 2-3 (Comp.) | (3 × 10⁻⁴)% Lemon aspen | 2.00 | 0.20 | |
| | extract | | | |
| 2-4 (Comp.) | 0.1% Fermented Oat | 0.91 | 0.21 | |
| 2-5 (Comp.) | 0.5% Fermented Oat | 2.47 | 0.88 | |
| 2-6 | 0.1% Fermented Oat + | 1.27 | 0.17 | (0.91 + 1.13 − 1) = 1.04 |
| | (3 × 10⁻⁵)% Lemon aspen | | | 1.27 > 1.04 |
| | extract | | | |
| 2-7 | 0.1% Fermented Oat + | 4.15 | 1.11 | (0.91 + 2 − 1) = 1.91; |
| | (3 × 10⁻⁴)% Lemon aspen | | | 4.15 > 1.91 |
| | extract | | | |
| 2-8 | 0.5% Fermented Oat + | 7.61 | 3.31 | (2.47 + 1.13 − 1) = 2.6 |
| | (3 × 10⁻⁵)% Lemon aspen | | | 7.61 > 2.6 |
| | extract | | | |
| 2-9 | 0.5% Fermented Oat + | 7.49 | 4.53 | (2.47 + 2 − 1) = 3.47 |
| | (3 × 10⁻⁴)% Lemon aspen | | | 7.49 > 3.47 |
| | extract | | | |

TABLE 3
IL8 Average Fold Change over Untreated

| | | Average Fold | | |
|---|---|---|---|---|
| | | Change over | Standard | |
| Sample | Treatment | untreated | Deviation | Synergy Comparison |
| 2-1 (Comp.) | Untreated | 1.00 | 0.60 | |
| 2-2 (Comp.) | (3 × 10−5)% Lemon aspen | 2.37 | 1.19 | |
| | extract | | | |
| 2-3 (Comp.) | (3 × 10−4)% Lemon aspen | 6.35 | 2.30 | |
| | extract | | | |
| 2-4 (Comp.) | 0.1% Fermented Oat | 2.74 | 1.06 | |
| 2-5 (Comp.) | 0.5% Fermented Oat | 18.23 | 1.38 | |
| 2-6 | 0.1% Fermented Oat + | 2.56 | 1.00 | (2.74 + 2.37 − 1) = 4.11 |
| | (3 × 10−5)% Lemon aspen | | | 2.56 < 4.11 |
| | extract | | | |
| 2-7 | 0.1% Fermented Oat + | 5.84 | 1.22 | (2.74 + 6.32 − 1) = 8.09 |
| | (3 × 10−4)% Lemon aspen | | | 5.84 < 8.09 |
| | extract | | | |
| 2-8 | 0.5% Fermented Oat + | 17.41 | 10.54 | (18.23 + 2.37 − 1) = 19.6 |
| | (3 × 10−5)% Lemon aspen | | | 17.41 < 19.6 |
| | extract | | | |
| 2-9 | 0.5% Fermented Oat + | 39.93 | 9.95 | |
| | (3 × 10−4)% Lemon aspen | | | |
| | extract | | | |

TABLE 4
HAS2 Average Fold Change over Untreated

| | | Average Fold | |
|---|---|---|---|
| | | Change over | Standard |
| Sample | Treatment | untreated | Deviation |
| 2-1 (Comp.) | Untreated | 1.00 | 0.16 |
| 2-2 (Comp.) | (3 × 10 − 5)% Lemon aspen | 0.68 | 0.09 |
| | extract | | |
| 2-3 (Comp.) | (3 × 10 − 4)% Lemon aspen | 0.73 | 0.08 |
| | extract | | |
| 2-4 (Comp.) | 0.1% Fermented Oat | 1.50 | 0.14 |
| 2-5 (Comp.) | 0.5% Fermented Oat | 3.87 | 0.21 |
| 2-6 | 0.1% Fermented Oat + | 1.43 | 0.20 |
| | (3 × 10 − 5)% Lemon aspen | | |
| | extract | | |
| 2-7 | 0.1% Fermented Oat + | 0.83 | 0.13 |
| | (3 × 10 − 4)% Lemon aspen | | |
| | extract | | |
| 2-8 | 0.5% Fermented Oat + | 2.17 | 1.18 |
| | (3 × 10 − 5)% Lemon aspen | | |
| | extract | | |
| 2-9 | 0.5% Fermented Oat + | 2.38 | 0.56 |
| | (3 × 10 − 4)% Lemon aspen | | |
| | extract | | |

With respect to CRABP2, not only did the fermented oat and lemon aspen extract complement each other in terms of bioactivity, but surprisingly did so synergistically. The most pronounced synergies were seen for fermented oat in combination with lemon aspen extract in Samples 2-7, 2-8 and 2-9. CRABP2 is well-established in literature as a highly sensitive marker of retinoid bioactivity and potency (Elder J T, Cromie M A, Griffiths C E M, Chambon P, Voorhees J J. Stimulus-selective induction of CRABP-II mRNA: A marker for retinoic acid action in human skin. J Invest Dermatol. 1993; 100(4), and has been associated with conferring skin anti-aging benefits (Bielli A, Scioli M G, D'Amico F, Tarquini C, Agostinelli S, Costanza G, Doldo E, Campione E, Passeri D, Coniglione F, Orlandi A. Cellular retinoic acid binding protein-II expression and its potential role in skin aging. Aging (Albany NY). 2019 Mar. 18; 11(6): 1619-1632). This data shows the combination of these two ingredients results in a synergistic increase of retinol-like property, and hence antiaging benefits. All synergistic results were statistically significant in this Example.

Similarly, a decreased synergy in terms of IL8 was observed in most combinations, particularly for Samples 2-6, 2-7 and 2-8. Retinoids are known to result in irritation and stinging upon application, which in vitro would be seen as IL8 induction. Thus, a reduction in IL8 corresponds to a clinical result of less irritation. The results indicated that even though the combination resulted in increased retinol-like activity, it did so without the same degree of inflammation side-effect. HAS2 is the hyaluronic acid synthase gene that produces hyaluronic acid, which is associated with skin hydration and plumping and is indirectly induced by retinol. Skin plumpness confers antiaging or youthful appearance. The samples did not demonstrate a hyaluronic acid synthase driven hydration synergy in the same manner as CRABP2 and IL8.

### Example 3: Bioactivity Assay of CRABP2 and IL8 in Human Dermal Fibroblasts (Various Oats and Lemon Aspen)

CRAPB2 and IL8 were evaluated in response to treatment with several types of processed oat products, Lemon aspen extract, and combinations of the two in varying amounts. Fermented oat test samples were prepared as described in Example 2. Colloidal oat (Avena Sativa Kernel Flour, Oat Cosmetics, Southampton, United Kingdom), was dissolved 100 mg in 1.0 mL media for a concentration of 10% 2 hours before use, then the 10% oat solution was sonicated for 10 minutes and centrifuged for 5 minutes. The Colloidal oat solution was further diluted to prepare 0.02%, 0.2% and 1% solutions. Avena sativa Oat Kernel extract (0.55% Avena sativa Kernel extract, 25% glycerin, 25% water) (Dragocalm®, Symrise, Holzminden, Germany) was diluted to prepare 0.01, 0.1 and 0.5% solutions in media. 100 μL of each test solution were added to each well to achieve the final concentrations as listed in Table 5-6 below. Results are shown as Average Fold Change over Untreated in Tables 5-6.

TABLE 5
CRABP2 Average Fold Change over Untreated

| | | Avg. Fold | | |
|---|---|---|---|---|
| | | Change over | Standard | Synergy |
| Sample | Treatment | untreated | Deviation | Comparison |
| 3-1 (Comp.) | Untreated | 1.00 | 0.07 | |
| 3-2 (Comp.) | 0.1% Fermented Oat | 1.42 | 0.46 | |
| 3-3 (Comp.) | 0.5% Fermented Oat | 1.24 | 0.33 | |
| 3-4 (Comp.) | 0.01% Oat extract | 0.95 | 0.10 | |
| 3-5 (Comp.) | 0.1% Oat extract | 0.90 | 0.23 | |
| 3-6 (Comp.) | 0.5% Oat extract | 0.98 | 0.03 | |
| 3-7 (Comp.) | 0.01% Colloidal oat | 1.02 | 0.13 | |
| 3-8 (Comp.) | 0.1% Colloidal oat | 1.10 | 0.44 | |
| 3-9 (Comp.) | 0.5% Colloidal oat | 2.74 | 0.87 | |
| 3-10 | (3 × 10 − 4)% Lemon aspen | 4.95 | 0.37 | (2.91 + 0.89 − 1) = 1.02 |
| | extract + 0.1% Oat extract | | | 4.95 > 1.02 |
| 3-11 | (3 × 10 − 4)% Lemon aspen | 5.26 | 1.31 | (2.91 + 0.98 − 1) = 2.89 |
| | extract + 0.5% Oat extract | | | 5.26 > 2.89 |
| 3-12 | (3 × 10 − 4)% Lemon aspen | 8.08 | 1.10 | (2.91 + 1.09 − 1) = 3 |
| | extract + 0.1% Colloidal oat | | | 8.07 > 3 |
| 3-13 | (3 × 10 − 4)% Lemon aspen | 6.33 | 1.28 | (2.91 + 2.74 − 1) = 4.65 |
| | extract + 0.5% Colloidal oat | | | 6.33 > 4.65 |
| 3-14 | (3 × 10 − 4)% Lemon aspen | 4.77 | 0.36 | (2.92 + 1.42 − 1) = 3.34 |
| | extract + 0.1% Fermented | | | 4.77 > 3.34 |
| | Oat | | | |
| 3-15 | (3 × 10 − 4)% Lemon aspen | 2.92 | 1.89 | |
| (Comp.) | extract | | | |

TABLE 6
IL8 Average Fold Change over Untreated

| | | Average Fold | | |
|---|---|---|---|---|
| | | Change over | Standard | Synergy |
| Sample | Treatment | untreated | Deviation | Comparison |
| 3-1 (Comp.) | Untreated | 1.00 | 0.05 | |
| 3-2 (Comp.) | 0.1% Fermented Oat | 2.64 | 0.92 | |
| 3-3 (Comp.) | 0.5% Fermented Oat | 5.16 | 5.09 | |
| 3-4 (Comp.) | 0.01% Oat extract | 1.09 | 0.19 | |
| 3-5 (Comp.) | 0.1% Oat extract | 2.21 | 1.34 | |
| 3-6 (Comp.) | 0.5% Oat extract | 1.24 | 0.53 | |
| 3-7 (Comp.) | 0.01% Colloidal oat | 3.54 | 2.62 | |
| 3-8 (Comp.) | 0.1% Colloidal oat | 3.58 | 1.21 | |
| 3-9 (Comp.) | 0.5% Colloidal oat | 7.19 | 5.17 | |
| 3-10 | (3 × 10 − 4)% Lemon | 8.61 | 2.19 | 4.86 + 2.21 − 1 = |
| | aspen extract + 0.1% | | | 6.07 |
| | Oat extract | | | |
| 3-11 | (3 × 10 − 4)% Lemon | 7.21 | 3.81 | 4.86 + 1.24 − 1 = |
| | aspen extract + 0.5% | | | 5.1 |
| | Oat extract | | | |
| 3-12 | (3 × 10 − 4)% Lemon | 17.88 | 6.82 | 4.86 + 3.58 − 1 = |
| | aspen extract + 0.1% | | | 7.44 |
| | Colloidal oat | | | |
| 3-13 | (3 × 10 − 4)% Lemon | 37.32 | 20.65 | 4.86 + 7.19 − 1 = |
| | aspen extract + 0.5% | | | 11.05 |
| | Colloidal oat | | | |
| 3-14 | (3 × 10 − 4)% Lemon | 11.09 | 1.76 | 4.86 + 2.64 − 1 = |
| | aspen extract + 0.1% | | | 6.5 |
| | Fermented Oat | | | |
| 3-15 (Comp.) | (3 × 10 − 4)% Lemon | 4.86 | 3.26 | |
| | aspen extract | | | |

The results show that all three forms of oat, colloidal oat, fermented oat and oat extract, when combined with lemon aspen extract synergistically increased retinoid activity, as measured by CRABP2. Again, as CRABP2 is a biomarker associated with anti-aging benefits, such a synergy is very advantageous.

### Example 4: Bioactivity Assay of CRABP2, HBEGF, IL8, and HAS3 in Whole Tissue Human Skin Explants of Samples with Fermented Oat and Lemon Aspen Extract, Alone and in Combination

Propylene glycol/ethanol (PGEtOH) (3:7) (w/w) was used as a vehicle. Lemon aspen extract and Fermented Oat materials, as described previously, were diluted in PGEtOH to the concentrations shown in Tables 7-10. Combinations were prepared by adding the appropriate amount of test solutions to arrive at the final concentrations as shown.

Abdominal skin samples were obtained from human adults undergoing abdominoplasty surgery. Informed consent was obtained from each patient, and all experimental steps were approved by an institutional review board (IRB). Subcutaneous fat was carefully removed and skin biopsies of 0.93 cm² were prepared under sterile conditions and acclimated in DMEM/F12 (1:1) medium, 2% heat-inactivated fetal bovine serum, 10 μg/mL insulin, 10 ng/mL hydrocortisone, 10 ng/mL EGF, 1×ABAM under a 5% CO₂ humidified atmosphere overnight. Skin explants were treated topically with 4 μL of each formulation for 48 hours.

At the end of the 48 h incubation, the skin biopsies were cut in half and either half of the skin biopsies or the two-halves were each lysed in 600 μL lysis buffer, consisting of 100 parts RLT buffer (RNA purification kit, sold under the tradename RNEASY Mini kit, Qiagen, Valencia, CA), to one part 2-mercaptoethanol inside a reinforced tube with screw cap and o-ring closure, and ceramic beads in the tube for tissue grinding (sold under the tradename PRECELLYS CKMix50-R, Bertin Corp, Rockville, MD). The tubes were shaken 40 seconds at 6300 rpm. RNA was extracted from the solutions using the RNEASY Mini Kit (Qiagen, Valencia, CA) according to manufacturer's instructions and RNA was eluted in 30 μL RNase-free water

Reverse transcription (RT) was performed using the Applied Biosystems High Capacity Reverse Transcription Kit (ThermoFisher Scientific, Bridgewater, NJ). Gene expression assays sold under the tradename TAQMAN for cellular retinoic acid binding protein 2 (CRABP2), heparin-binding epidermal growth factor (HbEGF), hyaluronic acid 15 synthase 3 (HAS3), interleukin-8 (IL8), polymerase (RNA) II polypeptide A (POLR2A), 18S rRNA (18S), TATA box binding protein (TBP), and master mix sold under the tradename TAQMAN (ThermoFisher Scientific, Bridgewater, NJ). qPCR analysis was performed using the TAQMAN master mix, and run on a real time PCR system sold under the tradename QUANTSTUDIO 7 Flex System (ThermoFisher Scientific, Bridgewater, NJ). The expression of these genes was normalized against either the expression of the human POLR2A, or alternatively against the expression of the human 18S rRNA or TBP “housekeeping” genes. These “housekeeping” genes are widely used controls for normalizing specific gene expression levels because of their invariant expression across tissues, cells, and experimental treatments. The method for normalization involves measuring the expression of an internal reference or “housekeeping” gene, which takes into account the potential error of RNA/cDNA loading, and variation of reverse transcription efficiency. The fold changes were calculated in comparison to the untreated or vehicle controls and two-tailed two-sample Student t-tests (Microsoft Office Excel 2007; Microsoft, Redmond, WA, USA) were performed. Synergies were calculated as described above. Results are shown as Average Fold Change over Untreated in Tables 7-10.

TABLE 7
CRABP2 Average Fold Change over Untreated

| | | Avg. Fold | | |
|---|---|---|---|---|
| | | Change over | Std. | Synergy |
| Sample | Treatment | untreated | Dev. | Comparison |
| 4-1 (Comp.) | Untreated | 1.00 | 0.12 | |
| 4-2 (Comp.) | PGEtOH | 0.77 | 0.20 | |
| 4-3 (Comp.) | 0.25% Lemon aspen extract | 1.51 | 0.50 | |
| 4-4 (Comp.) | 0.5% Lemon aspen extract | 1.58 | 0.86 | |
| 4-5 (Comp.) | 1% Lemon aspen extract | 1.99 | 0.69 | |
| 4-6 (Comp.) | 0.5% Fermented Oat | 1.00 | 0.23 | |
| 4-7 (Comp.) | 2% Fermented Oat | 1.01 | 0.13 | |
| 4-8 (Comp.) | 5% Fermented Oat | 0.94 | 0.20 | |
| 4-9 (Comp.) | 10% Fermented Oat | 0.98 | 0.14 | |
| 4-10 | 0.25% Lemon aspen extract + | 1.10 | 0.18 | |
| | 2% Fermented Oat | | | |
| 4-11 | 0.25% Lemon aspen extract + | 1.74 | 0.91 | (1.51 + 0.94 − 1) = 1.45 |
| | 5% Fermented Oat | | | 1.74 > 1.45 |
| 4-12 | 0.5% Lemon aspen extract + | 2.11 | 0.76 | (1.58 + 1.00 − 1) = 1.58 |
| | 0.5% Fermented Oat | | | 2.11 > 1.58 |
| 4-13 | 0.5% Lemon aspen extract + | 1.73 | 0.80 | (1.58 + 1.01 − 1) = 1.59 |
| | 2% Fermented Oat | | | 1.73 > 1.59 |
| 4-14 | 0.5% Lemon aspen extract + | 1.83 | 0.60 | (1.58 + 0.94 − 1) = 1.52 |
| | 5% Fermented Oat | | | 1.83 > 1.52 |

TABLE 8
HAS3 Average Fold Change over Untreated

| | | Avg. Fold | | |
|---|---|---|---|---|
| | | Change over | Standard | Synergy |
| Sample | Treatment | untreated | Deviation | Comparison |
| 4-1 (Comp.) | Untreated | 1.00 | 0.44 | |
| 4-2 (Comp.) | PGEtOH | 0.79 | 0.22 | |
| 4-3 (Comp.) | 0.25% Lemon aspen | 1.52 | 0.78 | |
| | extract | | | |
| 4-4 (Comp.) | 0.5% Lemon aspen | 1.34 | 0.69 | |
| | extract | | | |
| 4-5 (Comp.) | 1% Lemon aspen extract | 2.21 | 1.06 | |
| 4-6 (Comp.) | 0.5% Fermented Oat | 0.90 | 0.28 | |
| 4-7 (Comp.) | 2% Fermented Oat | 1.05 | 0.29 | |
| 4-8 (Comp.) | 5% Fermented Oat | 0.89 | 0.23 | |
| 4-9 (Comp.) | 10% Fermented Oat | 1.23 | 0.40 | |
| 4-10 | 0.25% Lemon aspen | 1.10 | 0.31 | |
| | extract + 2% Fermented | | | |
| | Oat | | | |
| 4-11 | 0.25% Lemon aspen | 2.01 | 1.48 | (1.52 + 0.89 − 1) = 1.41 |
| | extract + 5% Fermented | | | 2.01 > 1.41 |
| | Oat | | | |
| 4-12 | 0.5% Lemon aspen | 1.92 | 1.12 | (1.34 + 0.90 − 1) = 1.24 |
| | extract + 0.5% | | | 1.92 > 1.24 |
| | Fermented Oat | | | |
| 4-13 | 0.5% Lemon aspen | 1.97 | 1.29 | (1.34 + 1.05 − 1) = 1.39 |
| | extract + 2% Fermented | | | 1.97 > 1.39 |
| | Oat | | | |
| 4-14 | 0.5% LA + 5% | 2.13 | 0.90 | (1.34 + 0.89 − 1) = 1.23 |
| | Fermented Oat | | | 2.13 > 1.23 |

TABLE 9
HBEGF Average Fold Change over Untreated

| | | Avg. Fold | | |
|---|---|---|---|---|
| | | Change over | Std. | Synergy |
| Sample | Treatment | untreated | Dev. | Comparison |
| 4-1 (Comp.) | Untreated | 1.00 | 0.20 | |
| 4-2 (Comp.) | PGEtOH | 0.97 | 0.28 | |
| 4-3 (Comp.) | 0.25% Lemon aspen | 1.75 | 0.67 | |
| | extract | | | |
| 4-4 (Comp.) | 0.5% Lemon aspen extract | 1.86 | 0.92 | |
| 4-5 (Comp.) | 1% Lemon aspen extract | 3.08 | 1.40 | |
| 4-6 (Comp.) | 0.5% Fermented Oat | 1.31 | 0.29 | |
| 4-7 (Comp.) | 2% Fermented Oat | 1.32 | 0.16 | |
| 4-8 (Comp.) | 5% Fermented Oat | 1.33 | 0.23 | |
| 4-9 (Comp.) | 10% Fermented Oat | 1.52 | 0.20 | |
| 4-10 | 0.25% Lemon aspen | 1.39 | 0.38 | |
| | extract + 2% Fermented | | | |
| | Oat | | | |
| 4-11 | 0.25% Lemon aspen | 2.37 | 1.68 | (1.75 + 1.33 − 1) = 2.08 |
| | extract + 5% Fermented | | | 2.37 > 2.08 |
| | Oat | | | |
| 4-12 | 0.5% Lemon aspen extract + | 2.58 | 1.27 | (1.75 + 1.31 − 1) = 2.17 |
| | 0.5% Fermented Oat | | | 2.58 > 2.17 |
| 4-13 | 0.5% Lemon aspen extract + | 2.44 | 1.20 | (1.86 + 1.32 − 1) = 2.18 |
| | 2% Fermented Oat | | | 2.44 > 2.18 |
| 4-14 | 0.5% Lemon aspen extract + | 2.80 | 0.82 | (1.86 + 1.33 − 1) = 2.19 |
| | 5% Fermented Oat | | | 2.80 > 2.19 |

TABLE 10
IL8 Average Fold Change over Untreated

| | | Avg. Fold | | |
|---|---|---|---|---|
| | | Change over | Std. | Synergy |
| Sample | Treatment IL8 | untreated | Dev. | Comparison |
| 4-1 (Comp.) | Untreated | 1.00 | 0.40 | |
| 4-2 (Comp.) | PGEtOH | 1.11 | 0.29 | |
| 4-3 (Comp.) | 0.25% Lemon aspen extract | 1.68 | 0.36 | |
| 4-4 (Comp.) | 0.5% Lemon aspen extract | 2.39 | 0.79 | |
| 4-5 (Comp.) | 1% Lemon aspen extract | 2.73 | 1.25 | |
| 4-6 (Comp.) | 0.5% Fermented Oat | 1.26 | 0.15 | |
| 4-7 (Comp.) | 2% Fermented Oat | 1.01 | 0.20 | |
| 4-8 (Comp.) | 5% Fermented Oat | 1.61 | 0.68 | |
| 4-9 (Comp.) | 10% Fermented Oat | 1.84 | 0.22 | |
| 4-10 | 0.25% Lemon aspen extract + | 1.69 | 0.40 | |
| | 2% Fermented Oat | | | |
| 4-11 | 0.25% Lemon aspen | 2.41 | 1.23 | |
| | extract + 5% Fermented Oat | | | |
| 4-12 | 0.5% Lemon aspen extract + | 2.36 | 1.48 | (2.39 + 1.26 − 1) = 2.65 |
| | 0.5% Fermented Oat | | | 2.36 < 2.65 |
| 4-13 | 0.5% Lemon aspen extract + | 2.67 | 1.40 | |
| | 2% Fermented Oat | | | |
| 4-14 | 0.5% Lemon aspen extract + | 3.15 | 0.79 | |
| | 5% Fermented Oat | | | |

As can be seen from the above results for samples 4-11 to 4-14, fermented oat when combined with lemon aspen extract synergistically increased retinoid activity, as measured by CRABP2, HAS3, and HBEGF, biomarkers associated anti-aging benefits. Sample 4-12 displayed a synergistic decrease for IL-8.

### Example 5: Bioactivity Assay of CRABP2, HBEGE, IL8, and HAS3 in Human Skin Explants of Samples with Colloidal Oat and Lemon Aspen Extract, Alone and in Combination

Bioactivity assay was performed according to the methods described in Example 4 (normalized to TBP here), with the exception that prior to collection, topically treated explants were cut in half. One-half of each tissue was used to measure gene expression levels in the whole tissue, and in the other half-tissue, the tissue was separated into its dermis and epidermis by putting the tissue in a tube containing 100 μL of water at 60° C. After 50 seconds of contact, the epidermis was detached from the dermis and transferred separately into hard tissue homogenizing Ozyme CK28 tubes (Bertin Corp, Rockville, MD) containing ceramic beads, 400 μL of RLT buffer, and 4 μL beta-mercaptoethanol and put in ice. For dermis material, 10 μL of Proteinase K was added in each tube, mixed, and incubated at 55° C. for 2 h. Gene expression was tested using whole tissue and dermal tissue. Dermal tissue being made of fibroblasts (in contrast to epidermal tissues being made of keratinocytes) compares more accurately with data obtained in the primary cultures of fibroblasts shown in Example 2. Lemon aspen extract test samples were prepared as described in Example 4. Colloidal oat (oat flour), was dissolved in PgETOH to prepare 1% and 3% solutions. Combinations were prepared by adding the appropriate amount of test solutions to arrive at the final concentrations as shown below. Average fold change and synergies were calculated as described above. Results are shown as Average Fold Change over Untreated in Tables 11-18.

TABLE 11
CRABP2 Average Fold Change over Untreated in Dermis

| | | CRABP2 | |
|---|---|---|---|
| | | Avg. Fold | |
| | | Change vs | Standard |
| Sample | Treatment | Untreated | Deviation |
| 5-1 (Comp.) | Untreated | 1.00 | 0.08 |
| 5-2 (Comp.) | PGEtOH | 0.91 | 0.19 |
| 5-3 (Comp.) | 0.25% Lemon aspen extract | 1.19 | 0.29 |
| 5-4 (Comp.) | 0.5% Lemon aspen extract | 1.16 | 0.38 |
| 5-5 (Comp.) | 1% Lemon aspen extract | 1.03 | 0.27 |
| 5-6 (Comp.) | 2% Lemon aspen extract | 1.23 | 0.36 |
| 5-7 (Comp.) | 1% Colloidal oat | 0.83 | 0.20 |
| 5-8 (Comp.) | 3% Colloidal oat | 1.09 | 0.28 |
| 5-9 | 1% Colloidal oat + 0.25% | 1.11 | 0.35 |
| | Lemon aspen extract | | |
| 5-10 | 1% Colloidal oat + 0.5% | 1.11 | 0.29 |
| | Lemon aspen extract | | |
| 5-11 | 3% Colloidal oat + 0.25% | 0.99 | 0.18 |
| | Lemon aspen extract | | |
| 5-12 | 3% Colloidal oat + 0.5% | 1.04 | 0.05 |
| | Lemon aspen extract | | |

TABLE 12
CRABP2 Average Fold Change over Untreated in Whole Tissue

| | | Avg. Fold | Std. | Synergy |
|---|---|---|---|---|
| Sample | Treatment | Change | Dev. | Comparison |
| 5-1 (Comp.) | Untreated | 1.01 | 0.12 | |
| 5-2 (Comp.) | PGEtOH | 0.89 | 0.27 | |
| 5-3 (Comp.) | 0.25% Lemon aspen extract | 1.76 | 0.30 | |
| 5-4 (Comp.) | 0.5% Lemon aspen extract | 2.05 | 0.32 | |
| 5-5 (Comp.) | 1% Lemon aspen extract | 2.87 | 0.71 | |
| 5-6 (Comp.) | 2% Lemon aspen extract | 3.97 | 0.38 | |
| 5-7 (Comp.) | 1% Colloidal oat | 1.20 | 0.27 | |
| 5-8 (Comp.) | 3% Colloidal oat | 1.83 | 1.01 | |
| 5-9 | 1% Colloidal oat + 0.25% | 2.50 | 0.87 | (1.20 + 1.76 − 1) = 1.96 |
| | Lemon aspen extract | | | 2.50 > 1.96 |
| 5-10 | 1% Colloidal oat + 0.5% | 1.80 | 0.18 | |
| | Lemon aspen extract | | | |
| 5-11 | 3% Colloidal oat + 0.25 Lemon | 1.37 | 0.39 | |
| | aspen extract | | | |
| 5-12 | 3% Colloidal oat + 0.5% | 2.23 | 0.19 | |
| | Lemon aspen extract | | | |

TABLE 13
HAS3 Average Fold Change over Untreated in Dermis

| | | CRABP2 | | |
|---|---|---|---|---|
| | | Avg. Fold | | |
| | | Change vs | Std. | Synergy |
| Sample | Treatment | Untreated | Dev. | Comparison |
| 5-1 (Comp.) | Untreated | 1.02 | 0.24 | |
| 5-2 (Comp.) | PGEtOH | 0.92 | 0.37 | |
| 5-3 (Comp.) | 0.25% Lemon aspen extract | 1.70 | 0.59 | |
| 5-4 (Comp.) | 0.5% Lemon aspen extract | 2.24 | 0.48 | |
| 5-5 (Comp.) | 1% Lemon aspen extract | 2.99 | 0.32 | |
| 5-6 (Comp.) | 2% Lemon aspen extract | 3.57 | 0.46 | |
| 5-7 (Comp.) | 1% Colloidal oat | 1.84 | 0.38 | |
| 5-8 (Comp.) | 3% Colloidal oat | 1.77 | 0.44 | |
| 5-9 | 1% Colloidal oat + 0.25% | 2.06 | 0.87 | |
| | Lemon aspen extract | | | |
| 5-10 | 1% Colloidal oat + 0.5% | 2.87 | 0.85 | |
| | Lemon aspen extract | | | |
| 5-11 | 3% Colloidal oat + 0.25% | 2.88 | 0.85 | (1.77 + 1.70 − 1) = 2.47 |
| | Lemon aspen extract | | | 2.88 > 2.47 |
| 5-12 | 3% Colloidal Oat + 0.5% | 3.43 | 0.50 | (1.77 + 2.24 − 1) = 3.01 |
| | Lemon aspen extract | | | 3.43 > 3.01 |

TABLE 14
HAS3 Average Fold Change over Untreated in Whole Tissue

| | | CRABP2 | | |
|---|---|---|---|---|
| | | Avg. Fold | | |
| | | Change vs | Std. | Synergy |
| Sample | Treatment | Untreated | Dev. | Comparison |
| 5-1 (Comp.) | Untreated | 1.00 | 0.08 | |
| 5-2 (Comp.) | PGEtOH | 0.94 | 0.36 | |
| 5-3 (Comp.) | 0.25% Lemon aspen extract | 1.23 | 0.12 | |
| 5-4 (Comp.) | 0.5% Lemon aspen extract | 1.36 | 0.05 | |
| 5-5 (Comp.) | 1% Lemon aspen extract | 1.74 | 0.39 | |
| 5-6 (Comp.) | 2% Lemon aspen extract | 2.36 | 0.70 | |
| 5-7 (Comp.) | 1% Colloidal Oat | 1.41 | 0.18 | |
| 5-8 (Comp.) | 3% Colloidal Oat | 1.58 | 0.55 | |
| 5-9 | 1% Colloidal Oat + 0.25% | 2.28 | 0.80 | (1.41 + 1.23 − 1) = 1.64 |
| | Lemon aspen extract | | | 2.28 > 1.64 |
| 5-10 | 1% Colloidal Oat + 0.5% | 1.37 | 0.35 | |
| | Lemon aspen extract | | | |
| 5-11 | 3% Colloidal Oat + 0.25% | 1.34 | 0.39 | |
| | Lemon aspen extract | | | |
| 5-12 | 3% Colloidal Oat + 0.5% | 1.74 | 0.54 | |
| | Lemon aspen extract | | | |

TABLE 15
HBEGF Average Fold Change over Untreated in Dermis

| | | CRABP2 | | |
|---|---|---|---|---|
| | | Avg. Fold | | |
| | | Change vs | Std. | Synergy |
| Sample | Treatment | Untreated | Dev. | Comparison |
| 5-1 (Comp.) | Untreated | 1.00 | 0.07 | |
| 5-2 (Comp.) | PGEtOH | 1.12 | 0.14 | |
| 5-3 (Comp.) | 0.25% Lemon aspen extract | 1.30 | 0.14 | |
| 5-4 (Comp.) | 0.5% Lemon aspen extract | 1.46 | 0.15 | |
| 5-5 (Comp.) | 1% Lemon aspen extract | 2.37 | 0.37 | |
| 5-6 (Comp.) | 2% Lemon aspen extract | 2.54 | 0.50 | |
| 5-7 (Comp.) | 1% Colloidal Oat | 1.14 | 0.32 | |
| 5-8 (Comp.) | 3% Colloidal Oat | 1.33 | 0.28 | |
| 5-9 | 1% Colloidal Oat + 0.25% | 1.21 | 0.43 | |
| | Lemon aspen extract | | | |
| 5-10 | 1% Colloidal Oat + 0.5% | 2.84 | 0.56 | (1.14 + 1.46 − 1) = 1.6 |
| | Lemon aspen extract | | | 2.84 > 1.6 |
| 5-11 | 3% Colloidal Oat + 0.25% | 2.69 | 0.51 | (1.33 + 1.30 − 1) = 1.63 |
| | Lemon aspen extract | | | 2.69 > 1.63 |
| 5-12 | 3% Colloidal Oat + 0.5% | 2.65 | 0.21 | (1.33 + 1.46 − 1) = 1.79 |
| | Lemon aspen extract | | | 2.65 > 1.79 |

TABLE 16
HBEGF Average Fold Change over Untreated in Whole Tissue

| | | CRABP2 | | |
|---|---|---|---|---|
| | | Avg. Fold | | |
| | | Change vs | Std. | Synergy |
| Sample | Treatment | Untreated | Dev. | Comparison |
| 5-1 (Comp.) | Untreated | 1.00 | 0.08 | |
| 5-2 (Comp.) | pgetoh | 1.06 | 0.33 | |
| 5-3 (Comp.) | 0.25% Lemon aspen extract | 1.56 | 0.18 | |
| 5-4 (Comp.) | 0.5% Lemon aspen extract | 1.85 | 0.10 | |
| 5-5 (Comp.) | 1% Lemon aspen extract | 2.10 | 0.36 | |
| 5-6 (Comp.) | 2% Lemon aspen extract | 2.58 | 0.34 | |
| 5-7 (Comp.) | 1% Colloidal Oat | 1.70 | 0.20 | |
| 5-8 (Comp.) | 3% Colloidal Oat | 1.94 | 1.03 | |
| 5-9 | 1% Colloidal Oat + 0.25% | 2.34 | 0.86 | (1.70 + 1.56 − 1) = 2.26 |
| | Lemon aspen extract | | | 2.34 > 2.26 |
| 5-10 | 1% Colloidal Oat + 0.5% | 1.83 | 0.34 | |
| | Lemon aspen extract | | | |
| 5-11 | 3% Colloidal Oat + 0.25% | 1.37 | 0.71 | |
| | Lemon aspen extract | | | |
| 5-12 | 3% Colloidal Oat + 0.5% | 1.92 | 0.40 | |
| | Lemon aspen extract | | | |

TABLE 17
IL8 Average Fold Change over Untreated in Dermis

| | | Avg. Fold | |
|---|---|---|---|
| | | Change vs | Standard |
| Sample | Treatment | Untreated | Deviation |
| 5-1 (Comp.) | Untreated | 1.18 | 0.86 |
| 5-2 (Comp.) | pgetoh | 1.31 | 0.55 |
| 5-3 (Comp.) | 0.25% Lemon aspen extract | 0.95 | 0.78 |
| 5-4 (Comp.) | 0.5% Lemon aspen extract | 1.12 | 0.55 |
| 5-5 (Comp.) | 1% Lemon aspen extract | 1.26 | 0.83 |
| 5-6 (Comp.) | 2% Lemon aspen extract | 0.91 | 0.80 |
| 5-7 (Comp.) | 1% Colloidal Oat | 1.16 | 0.53 |
| 5-8 (Comp.) | 3% Colloidal Oat | 1.47 | 0.48 |
| 5-9 | 1% Colloidal Oat + 0.25% | 1.31 | 0.76 |
| | Lemon aspen extract | | |
| 5-10 | 1% Colloidal Oat + 0.5% | 1.09 | 0.49 |
| | Lemon aspen extract | | |
| 5-11 | 3% Colloidal Oat + 0.25% | 1.74 | 0.94 |
| | Lemon aspen extract | | |
| 5-12 | 3% Colloidal Oat + 0.5% | 1.45 | 0.29 |
| | Lemon aspen extract | | |

TABLE 18
IL8 Average Fold Change over Untreated in Whole Tissue

| | | CRABP2 | | |
|---|---|---|---|---|
| | | Avg. Fold | | |
| | | Change vs | Std. | Synergy |
| Sample | Treatment | Untreated | Dev. | Comparison |
| 5-1 (Comp.) | Untreated | 1.04 | 0.33 | |
| 5-2 (Comp.) | pgetoh | 0.74 | 0.20 | |
| 5-3 (Comp.) | 0.25% Lemon aspen extract | 0.57 | 0.15 | |
| 5-4 (Comp.) | 0.5% Lemon aspen extract | 0.87 | 0.39 | |
| 5-5 (Comp.) | 1% Lemon aspen extract | 1.22 | 0.63 | |
| 5-6 (Comp.) | 2% Lemon aspen extract | 0.94 | 0.56 | |
| 5-7 (Comp.) | 1% Colloidal Oat | 1.10 | 1.00 | |
| 5-8 (Comp.) | 3% Colloidal Oat | 0.98 | 0.37 | |
| 5-9 | 1% Colloidal Oat + 0.25% | 0.84 | 0.19 | |
| | Lemon aspen extract | | | |
| 5-10 | 1% Colloidal Oat + 0.5% | 0.63 | 0.08 | (1.10 + 0.87 − 1) = 0.97 |
| | Lemon aspen extract | | | 0.63 < 0.97 |
| 5-11 | 3% Colloidal Oat + 0.25% | 1.15 | 0.64 | |
| | Lemon aspen extract | | | |
| 5-12 | 3% Colloidal Oat + 0.5% | 1.07 | 0.15 | |
| | Lemon aspen extract | | | |

EXAMPLE 5 uses a human skin explant model to validate synergy ex vivo. The dosages used reflect more similarly in vivo dosage use.

HBEGF induction was statistically significant over untreated for all oat plus lemon aspen extract combinations except for 3% colloidal oat plus 0.25% lemon aspen extract in whole tissue. 1% colloidal oat plus 0.25% lemon aspen extract was also comparable to 2% lemon aspen extract. In the dermis, HBEGF induction was statistically significant over untreated for all oat plus LA combinations except for 1% colloidal oat plus 0.25% lemon aspen extract. These results were consistent with the fibroblast data above.

1% Colloidal oat plus Lemon aspen extract combinations showed a statistically significant induction of CRABP2 over untreated in whole tissue. There were no statistically significant differences between treatments for CRABP2 in the dermis. CRABP2 did not change compared to untreated, indicating less sensitivity or response in this particular marker in contrast to the whole tissue.

IL8 also did not show significant induction as compared to untreated in response to any treatment, further highlighting the gentle nature of these ingredients and combination. The IL8 data exhibited too much variability to determine synergies, as observed by high levels of standard deviation.

1% Colloidal oat plus 0.25% lemon aspen extract and 3% Colloidal oat plus 0.5% lemon aspen extract showed a statistically significant induction of HAS3 over untreated in whole tissue. Additionally, 1% Colloidal oat plus 0.25% lemon aspen extract showed a statistically significant induction of HAS3 over 0.25% lemon aspen extract in whole tissue.

In the dermis, 0.25% lemon aspen extract and had comparable induction of HAS3 to 1% and 2% lemon aspen extract. Furthermore, 3% Colloidal oat plus 0.5% lemon aspen extract had a statistically significantly higher induction of HAS3 when compared to 0.5% lemon aspen extract alone.

What is claimed is:

1. A skincare composition comprising:
a. a compound of Formula I: 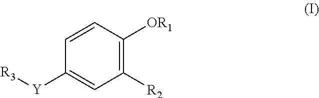
wherein:
R₁ is selected from the group consisting of C₁-C₂₀ alkyl, C₂-C₂₀ alkenyl, C₂-C₂₀ alkynyl, and C₃-C₈ cycloalkyl or aryl;
R₂ is selected from the group consisting of hydrogen, hydroxyl, C₁-C₆ alkyl, C₂-C₆ alkenyl, C₂-C₆ alkynyl, C₃-C₈ cycloalkyl or aryl, —OC₁-C₆ alkyl, —OC₂-C₆ alkenyl, —OC₂-C₆ alkynyl, —OC₃-C₈ cycloalkyl or aryl, thiol, —SC₁-C₆ alkyl, —SC₂-C₆ alkenyl, —SC₂-C₆ alkynyl, —SC₃-C₈ cycloalkyl or aryl, —NR₄C₁-C₆ alkyl, —NR₄C₂-C₆ alkenyl, —NR₄C₂-C₆ alkynyl, and —NR₄C₃-C₈ cycloalkyl or aryl;
R₃ is selected from —CO₂H, —CO₂R₄ or an isosteric equivalent of a carboxy group, wherein R⁴ is C₁-C₆ alkyl, C₂-C₆ alkenyl, C₃-C₈ cycloalkyl or aryl; and
Y is —(CH₂—CH₂)—, —(CH═CH)—, or —(C≡C)—;
or a cosmetically acceptable salt thereof; and
b. a processed oat ingredient.

2. The skincare composition of claim 1, wherein R¹ is selected from the group consisting of C₅-C₁₆ alkyl, C₅-C₁₆ alkenyl, and C₅-C₁₆ alkynyl; R₂ is selected from the group consisting of hydrogen, hydroxyl, —OC₁-C₆ alkyl, —OC₂-C₆ alkenyl, —OC₂-C₆ alkynyl, —OC₃-C₈ cycloalkyl; R₃ is selected from —CO₂H, —CO₂R₄ wherein R⁴ is C₁-C₆ alkyl, or an isosteric equivalent of a carboxy group; and Y is —(CH₂—CH₂)— or —(CH═CH)—.

3. The skincare composition of claim 1, wherein R¹ is selected from the group consisting of C₅-C₁₆ alkenyl; and R² is selected from the group consisting of hydrogen or —OC₁-C₃ alkyl.

4. The skincare composition of claim 1, wherein the compound of Formula I is selected from the group consisting of 3-(4-farnesyloxyphenyl)-propionic acid, 3-(4-farnesyloxy-3-hydroxyphenyl)-propionic acid, 3-(4-farnesyloxy-3-methoxyphenyl)-propionic acid, ethyl esters thereof, and combinations of two or more thereof.

5. The skincare composition of claim 4, wherein the compound of Formula I is comprises 3-(4-farnesyloxyphenyl)-propionic acid.

6. The skincare composition of claim 1, wherein the concentration of the compound of Formula I is present in an amount ranging from about 0.00001% to 10%, by total weight of the composition.

7. The skincare composition of claim 1, wherein the skincare composition comprises a botanical extract comprising the compound of Formula I.

8. The skincare composition of claim 7, wherein the botanical extract comprises an extract of a plant of the genus Acronychia.

9. The skincare composition of claim 8, wherein said botanical extract is an extract of Acronychia acidula.

10. The skincare composition of claim 9, wherein said botanical extract is a polar extract.

11. The skincare composition of claim 7, wherein the botanical extract is present in an amount ranging from about 0.00001% to about 5% by total weight of the composition.

12. The skincare composition of claim 1, wherein the extract of Acronychia acidula comprises 3-(4-farnesyloxyphenyl)-propionic acid in a concentration ranging from about about 0.1% to about 30% by total weight of the extract.

13. The skincare composition of claim 1, wherein the processed oat ingredient is selected from the group consisting of fermented oat, colloidal oat, oat extract, oat oil, and combinations thereof.

14. The skincare composition of claim 1, further comprising an ingredient selected from the group consisting of surfactants, chelating agents, emollients, humectants, conditioners, preservatives, opacifiers, fragrances, and combinations of two or more thereof.

15. The skincare composition of claim 1, wherein the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

16. The skincare composition of claim 1, wherein the compound of Formula I is comprises 3-(4-farnesyloxyphenyl)-propionic acid, and the processed oat ingredient is selected from the group consisting of fermented oat, oat extract, colloidal oat and combinations thereof.

17. A method for treating skin comprising topically applying the composition of claim 1.

18. The method of claim 17, wherein the method is a method for treating signs of aging, treating acne, smoothing skin texture, or brightening the skin.

19. The method of claim 17, wherein the method is a method of increasing CRABP2 expression.

20. A skincare composition comprising:
a. 3-(4-farnesyloxyphenyl)-propionic acid; and
b. a processed oat ingredient selected from the group consisting of fermented oat, colloidal oat, oat extract, oat oil, and combinations thereof,
wherein the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.
